SEQUENCE LISTING (6 sequences)

SEQ ID NO: 1 - protein, 317 aa, Homo sapiens
MKVLWAALLVTFLAGCQAKVKQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 2 - protein, 432 aa, Homo sapiens [sequence omitted]

SEQ ID NO: 3 - protein, 585 aa, Homo sapiens [sequence omitted]

SEQ ID NO: 4 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 5 - protein, 763 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQEGLGSPSARSPA

SEQ ID NO: 6 - protein, 302 aa, Homo sapiens [sequence omitted]

(19) United States
(12) Patent Application Publication
(10) Pub. No.: US 2015/0141276 A1
(43) Pub. Date: May 21, 2015

(54) IN VITRO METHOD FOR DIAGNOSIS OF ENDOMETRIOSIS

(71) Applicants: Pietro Giulio SIGNORILE, Roma (IT); Alfonso BALDI, Napoli (IT)

(72) Inventors: Pietro Giulio Signorile, Roma (IT); Alfonso Baldi, Napoli (IT)

(21) Appl. No.: 14/400,884

(22) PCT Filed: May 13, 2013
(86) PCT No.: PCT/IB2013/053875

§ 371 Date: Nov. 13, 2014

(30) Foreign Application Priority Data
May 14, 2012 (IT) RM2012A000214

(51) Int. Cl. G01N 33/68

(52) U.S. Cl. CPC G01N 33/689; G01N 2800/364; G01N 2333/4728
USPC 506/9; 436/501; 435/7.92; 435/23; 506/18

(57) ABSTRACT

The present description relates to an in vitro method for the diagnosis of endometriosis, an in vitro method for the monitoring of endometriosis and a kit for the diagnosis of endometriosis and/or for the monitoring of endometriosis in a subject.

The present description relates to an in vitro method for the diagnosis of endometriosis, an in vitro method for the monitoring of a therapy against endometriosis and a kit for the diagnosis of endometriosis and/or for the monitoring of a therapy against endometriosis in a subject.

### STATE OF THE PRIOR ART

Endometriosis is a pathological condition characterized by the presence of endometrial tissue outside the uterus, and is associated to pelvic pain and infertility (Giudice L C, and Kao L C: Endometriosis. The Lancet, 364: 1789-1799, 2004). Its incidence is estimated at about 10% of the female population of reproductive age (Houston D E: Evidence for the risk of pelvic endometriosis by age, race, and socioeconomic status. Epidemiol Rev, 6: 167-191, 1984.). The symptoms are, in general, not very specific, and often erroneously attributed to other pathologies causing chronic pains; therefore, a diagnosis of endometriosis is hardly performed at the earliest stages of the disease. Moreover, a diagnosis of certainty can be carried out only through an invasive approach, consisting in a laparoscopic inspection and histological analysis of suspect lesions. Therefore, the diagnosis of endometriosis is always posed with significant delay: current estimates indicate a time interval of 8-12 years between symptoms appearance and the diagnosis of endometriosis (Hadfield R, Mardon H, Barlow D, Kennedy S. Delay in the diagnosis of endometriosis: a survey of women from the USA and the UK. Hum Reprod 1996, 11: 878-880.). At present, there is no possibility of carrying out an early diagnosis of endometriosis through the use of less invasive methods. A recent review of the literature on the topic highlighted how, despite the great number of scientific publications on the topic, to date there is no biomarker or group of biomarkers that proved clinically effective to pose the diagnosis of endometriosis by use of non-invasive methodologies, like, for instance, a sample of peripheral blood (May K E, Conduit-Hulbert S A, Villar J, Kirtkley S, Kennedy S H, Becker C M: Peripheral biomarkers of endometriosis: a systematic review. Hum Reprod 2010, 16: 651-674). A very recent work by Fassbender et al. (Fassbender A, Waelkens E, Verbeeck N, Kyama C M, Bokor A, Vodolazkaia A, Van de Plas R, Meuleman C, Peeraer K, Tomassetti C, Gevaert O, Ojeda F, De Moor B, D'Hooghe T: Proteomics analysis of plasma for early diagnosis of endometriosis. Obstet Gynecol 2012, 119: correlate with the presence or absence of endometriosis. However, the authors, though observing a variation in peak patterns of about 10 different peptides/proteins in various stages of endometriosis (from minimal to severe stage) do not identify the origin of such proteins, which therefore remain unknown and, as such, not useful to define a group of biomarkers whose variation of expression may be considered advantageous for diagnostic purposes.

Hence, in the state of the known art the need of singling out non-invasive diagnostic instruments allowing an accurate, and possibly early, diagnosis of endometriosis in order to define a therapeutic strategy already in the early stages of disease development is highly felt.

### SUMMARY OF THE INVENTION

The present description relates to an in vitro method and a kit for the diagnosis of endometriosis.

The present invention is based on the discovery that the expression of a group of proteins, detailed in the next section, varies in a statistically significant manner in patients suffering from endometriosis with respect to the population of healthy controls.

In particular, the observation of the Inventors about the quantitative difference of the expression of the proteins at issue in subjects also in the earliest stages of the pathology with respect to healthy control patients, enabled to define for the first time a method for evaluating the presence of said pathology. One of the advantages of the present invention therefore consists in the possibility of reaching a diagnosis significantly ahead of the onset of symptoms typical of this pathology.

Therefore, the observation of the variation of the concentration of such proteins can be advantageously utilized for defining an in vitro method for the diagnosis of endometriosis in female subjects. Such variation of expression is observed particularly on blood samples of subjects with endometriosis. Therefore, even more advantageously, said method, in an embodiment thereof, is carried out in an absolutely noninvasive manner thanks to the fact that the determining of the concentration of the proteins of interest can be performed on a blood sample. In particular, taking into account that for a firm diagnosis of endometriosis currently methods are available which envisage invasive surgical practices aimed at obtaining a sample of endometrial tissue, it can be stated that, with respect to the state of the known art, in an embodiment, the diagnostic method described herein attains a considerable technical advancement consisting in a remarkable increase of compliance by subjects under examination.
Therefore, a first object of the present application is:

an in vitro method for the diagnosis of endometriosis in a subject, comprising the following steps:

a) determining the concentration of at least one protein comprising or consisting in a sequence selected from the group of: SEQ ID NO 1, SEQ ID NO 2, SEQ ID NO 3, SEQ ID NO 4, SEQ ID NO 5 and SEQ ID NO 6, or mutants and/or post-translational variants thereof, in a biological sample of said subject and in a control sample;

b) comparing said concentrations in said biological sample and said control sample

wherein an increase of the concentration of said at least one protein comprising or consisting in a sequence selected from the group of SEQ ID NO 2, SEQ ID NO 3, SEQ ID NO 6 and/or a decrease of the concentration of said at least one protein comprising or consisting in a sequence selected from the group of SEQ ID NO 1, SEQ ID NO 4, SEQ ID NO 5 with respect to the same protein in said control is indicative of endometriosis.

A second object of the present description is:

an in vitro method for the monitoring of a therapy against endometriosis in a subject under said therapy, comprising the following steps:

a) determining the concentration of at least one protein comprising or consisting in a sequence selected from the group of: SEQ ID NO 1, SEQ ID NO 2, SEQ ID NO 3, SEQ ID NO 4, SEQ ID NO 5 and SEQ ID NO 6, or mutants and/or post-translational variants thereof, in a first and in a second biological sample, obtained at different times, of said subject,

b) comparing said concentration obtained for said first and second sample.

Object of the present description is also a kit for the diagnosis of endometriosis and/or the monitoring of a therapy against endometriosis in a subject, comprising:

at least one aliquot of one or more reagents necessary to the determining of the concentration of at least one protein comprising or consisting in a sequence selected from the group of: SEQ ID NO 1, SEQ ID NO 2, SEQ ID NO 3, SEQ ID NO 4, SEQ ID NO 5 and SEQ ID NO 6 or mutants and/or post-translational variants thereof in a biological sample of said subject.

Further advantages, as well as the features and the operation steps of the present invention will be made apparent in the following detailed description of some preferred embodiments thereof, given merely by way of example and not for limitative purposes.

### DETAILED DESCRIPTION OF THE FIGURES

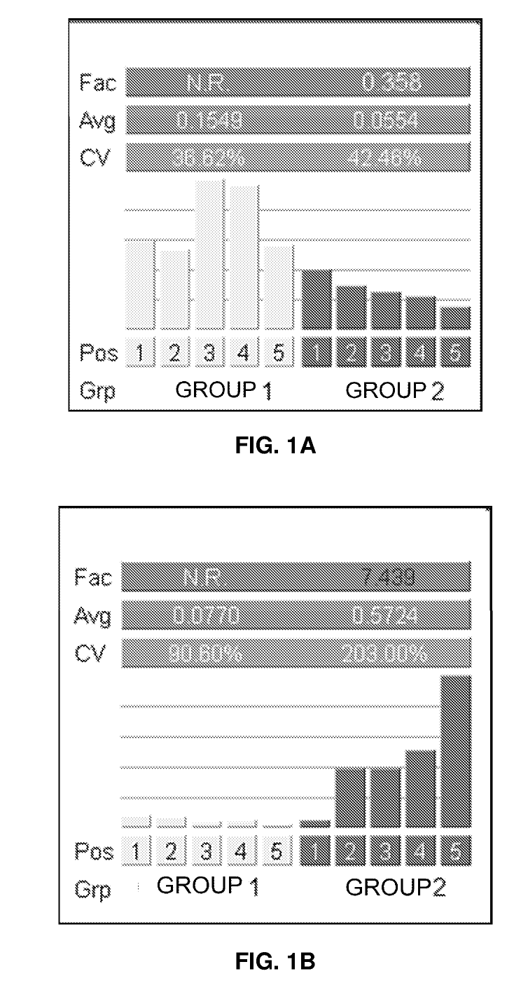

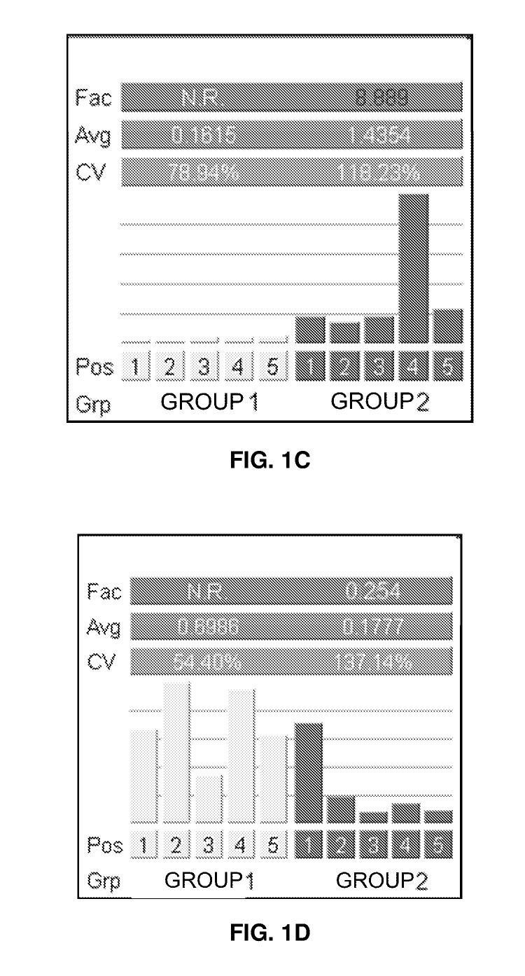

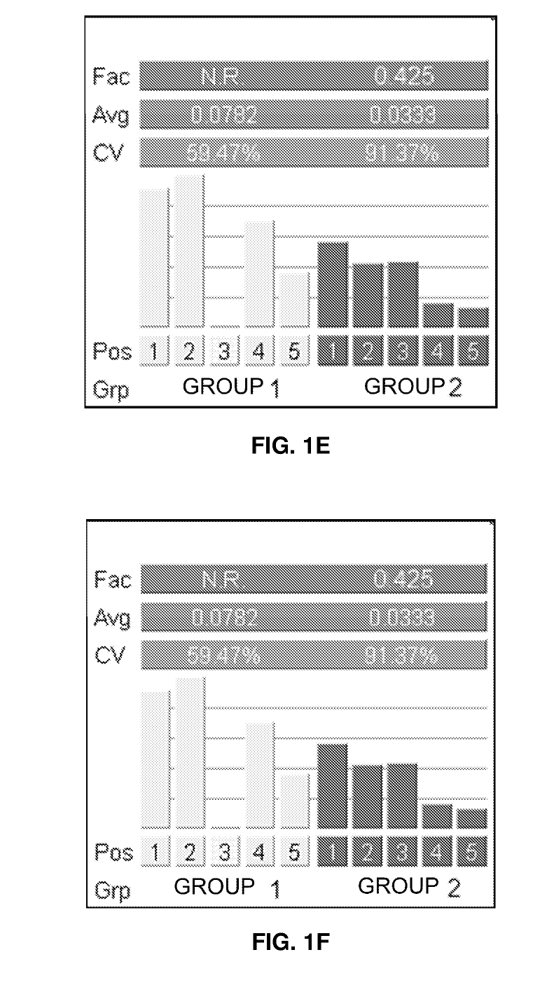

FIGS. 1A, 1B, 1C, 1D. 1E and 1F show histograms related to the different expression respectively of proteins of SEQ ID NO: 1, SEQ ID NO: 2, SEQ ID NO: 3, SEQ ID NO: 4, SEQ ID NO: 5 and SEQ ID NO: 6 in serum samples of patients with endometriosis (group 2) with respect to the serum samples of a control population (group 1). CV=variation coefficient, AVG=average value, Fac=regulation factor.

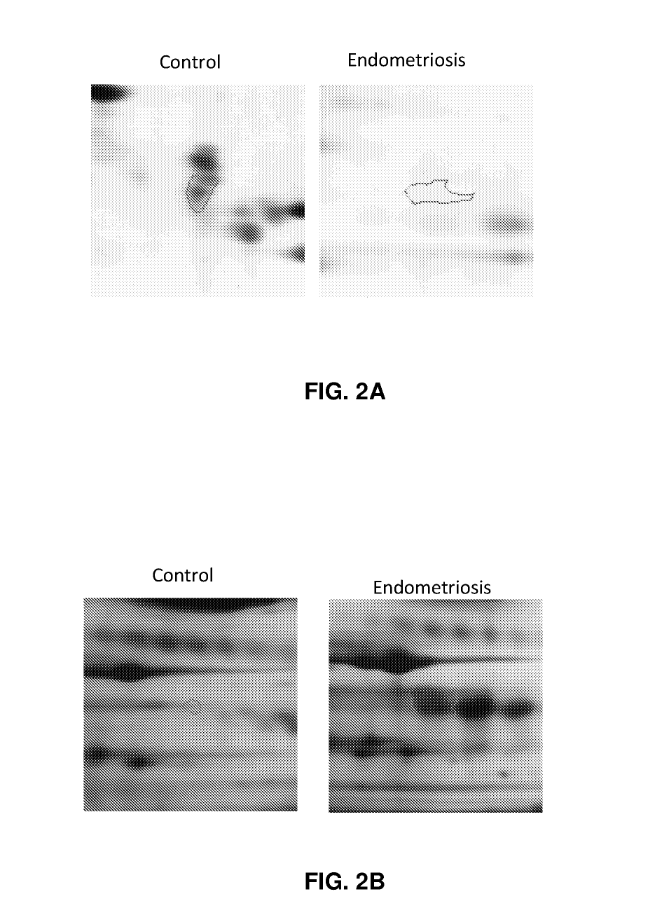

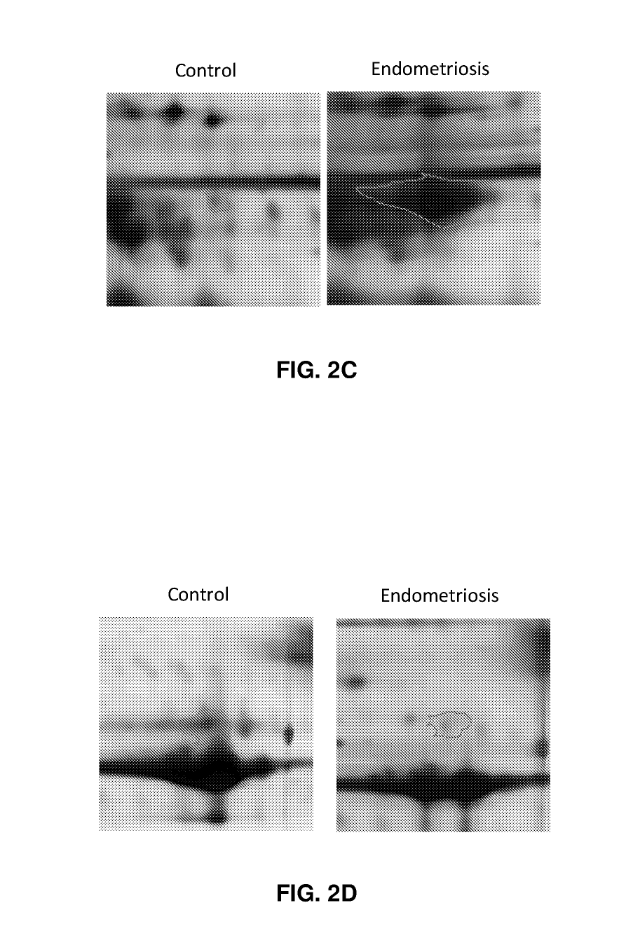

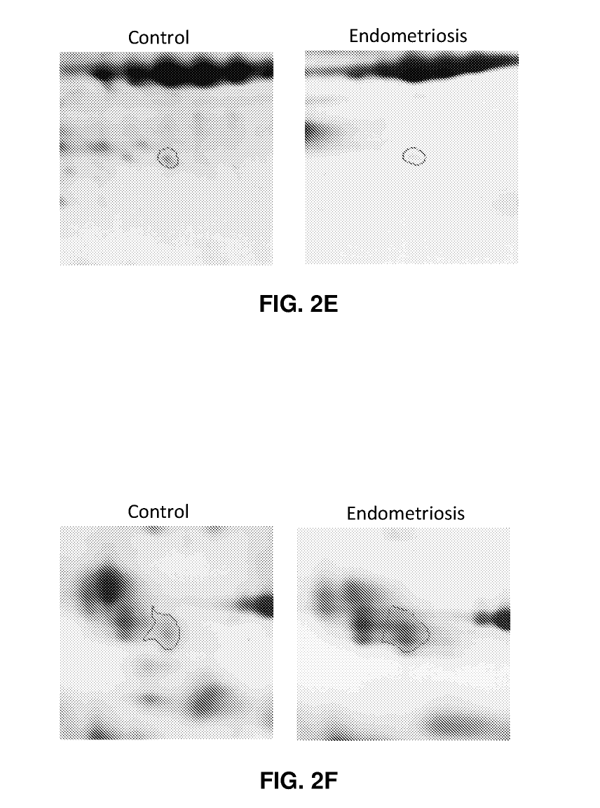

FIGS. 2A-F show a two-dimensional gel in which are visible the various expression levels of proteins of respectively SEQ ID NO: 1, SEQ ID NO: 2, SEQ ID NO: 3, SEQ ID NO: 4, SEQ ID NO: 5 and SEQ ID NO: 6 in control samples and in samples of patients with endometriosis.

### SEQUENCE DESCRIPTION

SEQ ID NO 1 Protein corresponding to human apolipoprotein E mutant E3K (Accession number in GenBank: GI 1506383A: 317 amino acids):

mkvlwaallv tflagcqakv kqavetepep elrqqtewqs
gqrwelalgr fwdylrwvqt lseqvqeell ssqvtqelra
lmdetmkelk aykseleeql tpvaeetrar lskelqaaqa
rlgadmedvc grlvqyrgev qamlgqstee lrvrlashlr
klrkrllrda ddlqkrlavy qagaregaer glsairerlg
plveqgrvra atvgslagqp lqeraqawge rlrarmeemg
srtrdrldev keqvaevrak leeqaqqirl qaeafqarlk
swfeplvedm qrqwaglvek vqaavgtsaa pvpsdnh.

SEQ ID NO 2 Protein corresponding to chain A of human antithrombin Iii complex (Accession number in GenBank: GI 999513 1ATH_A; 432 amino acids):

hgspvdicta kprdipmnpm ciyrspekka tedegseqki
peatnrrvwe lskansrfat tfyqhladsk ndndniflsp
lsistafamt klgacndtlq qlmevfkfdt isektsdqih
fffaklncrl yrkanksskl vsanrlfgdk sltfnetyqd
iselvygakl qpldfkenae qsraainkwv snktegritd
vipseainel tvlvlvntiy fkglwkskfs pentrkelfy
kadgescsas mmyqegkfry rrvaegtqvl elpfkgddit
mvlilpkpek slakvekelt pevlqewlde leemmlvvhm
prfriedgfs lkeqlqdmgl vdlfspeksk lpgivaegrd
dlyvsdafhk aflevneegs eaaastavvi agrslnpnrv
tfkanrpflv firevplnti ifmgrvanpc vk.

SEQ ID NO 3 Protein corresponding to chain A of human serum albumin (Accession number in GenBank: GI 122920512 212Z_A; 585 amino acids):

dahksevahr fkdlgeenfk alvliafaqy lqqcpfedhv
klvnevtefa ktcvadesae ncdkslhtlf gdklctvatl
retygemadc cakqeperne cflqhkddnp nlprlvrpev
dvmctafhdn eetflkkyly eiarrhpyfy apellffakr
ykaafteccq aadkaacllp kldelrdegk assakqrlxc
aslqkfgera fkawavarls qrfpkaefae vsklvtdltk
vhtecchgdl lecaddradl akyicenqds issklkecce
kpllekshci aevendempa dlpslaadfv eskdvcknya
eakdvflgmf lyeyarrhpd ysvvlllrla ktyettlekc
caaadphecy akvfdefkpl veepqnlikq ncelfeqlge
ykfqnallvr ytkkvpqvst ptivevsrnl gkvgskcckh
peakrmpcae dylsvvInql cvlhektpvs drvtkcctes
Ivnrrpcfsa levdetyvpk efnaetftfh adictlseke
rqikkqtalv elvkhkpkat keqlkavmdd faafvekcck
addketcfae egkklvaasq aalgl.

SEQ ID NO 4 Protein corresponding to complement C3 precursor [Homo sapiens]; Accession number in GenBank: GI 115298678 NP_{—}000055; 1663 amino acids):

mgptsgpsll llllthlpla lgspmysllt pnllrlesee
tmvleahdaq gdvpvtvtvh dfpgkklvls sektvltpat
nhmgnvtftl panrefksek grnkfvtvqa tfgtqvvekv
vlvslqsgyl flqtdktlyt pgstvlyrlf tvnhkllpvg
rtvmvnlenp eglpvkqdsl ssqnqlgvlp lswdlpelvn
mgqwklrayy enspqqvfst efevkeyvlp sfevlvepte
kfyylynekg levtltarfl ygkkvegtaf vlfglqdgeq
rlslpeslkr lpledgsgev vlsrkvlldg vqnpraedlv
gkslyvsatv llhsgsdmvq aersglplvt spyqlhftkt
pkyfkpgmpf dlmvfvtnpd gspayrvpva vqgedtvqsl
tqgdgvakls lnthpsqkpl sltvrtkkqe lseaeqatrt
mqalpystvg nsnnylhlsv lrtelrpget lnvnfllrmd
raheaklryy tyllmnkgrl lkagrqvrep gqdlvvlpls
lttdflpsfr lvayytllga sgqrevvads vwvdvkdscv
gslvvksgqs edrqpvpgqq mtlklegdhg arvvlvavdk
gvfvlnkknk ltqsklwdvv ekadlgctpg sgkdyagvfs
dagltftsss gqqtaqrael qcpqpaarrr rsvqltekrm
dkvgkypkel rkccedgmre npmrfscqrr trflslgeac
kkvfldccny ltelrrqhar ashlglarsn Ldedllaeen
lvsrsefpes wlwnvedlke ppknglstkl mnlflkdslt
twellaysms dkkglcvadp fevtvmqdff ldlrlpysvv
rneqvelrav lynyrqnqel kvrvellhnp afcslattkr
rhqqtvtlpp ksslsvpyvl vplktglqev evkaavyhhf
lsdgvrkslk vvpeglrmnk tvavrtldpe rlgregvqke
dlppadlsdq vpdtesetrl llqgtpvaqm tedavdaerl
khllvtpsgc geqnmlgmtp tvlavhylde teqwekfgle
krqgalellk kgytqqlafr qpssafaafv krapstwlta
yvvkvfslav nllaldsqvl cgavkwllle kqkpdgvfqe
dapvlhqeml gglrnnnekd maltafvlls lqeakdlcee
qvnslpgslt kagdfleany mnlqrsytva iagyalaqmg
rlkgpllnkf lttakdknrw edpgkqlynv eatsyallal
lqlkdfdfvp pvvrwlneqr yygggygstq atfmvfqala
qyqkdapdhq elnldvslql psrsskithr ihwesasllr
seetkenegf tvtaegkgqg tlsvvtmyha kakdqltcnk
fdlkvtikpa petekrpqda kntmileict ryrgdqdatm
sildismmtg fapdtddlkq langvdryis kyeldkafsd
rntliiyldk vshseddcla fkvhqyfnve liqpgavkvy
ayynleesct rfyhpekedg klnklcrdel crcaeencfi
qksddkvtle erldkacepg vdyvyktrlv kvqlsndfde
yimaieqtik sgsdevqvgq qrtfispikc realkleekk
hylmwglssd fwgekpnlsy iigkdtwveh wpeedecqde
enqkqcqdlg aftesmvvfg cpn.

SEQ ID NO 5 Protein isolated from Homo sapiens (Accession number in GenBank: GI 194380608 BAG58457; 763 amino acids):

mrllwgliwa ssfftlslqk prlllfspsv vhlgvplsvg
vqlqdvprgq vvkgsvflrn psrnnvpcsp kvdftlsser
dfallslqvp lkdakscglh qllrgpevql vahspwlkds
lsrttniqgi nllfssrrgh lflqtdqpiy npgqrvryry
faldqkmrps tdtitvmven shglrvrkke vympssifqd
dfvipdisep gtwkisarfs dglesnsstq fevkkyvlpn
fevkitpgkp yiltvpghld emqldiqary iygkpvqgva
yvrfgllded gkktffrgle sqtklvngqs hislskaefq
daleklnmgi tdlqglrlyv aaaiiespgg emeeaeltsw
yfvsspfsld lsktkrhlvp gapfllqalv remsgspasg
ipvkvsatvs spgsvpevqd iqqntdgsgq vsipiiipqt
iselqlsysa gsphpaiarl tvaappsggp gflsierpds
rpprvgdtln lnlravgsga tfshyyymil srgqivfmnr
epkrtltsys vfvdhhlaps fyfvafyyhg dhpvanslry
dvqagacegk lelsvdgakq yrngesvklh letdslalva
lgaldtalya agskshkpln mgkvfeamns ydlgcgpggg
dsalqvfqaa glafsdgdqw tlsrkrlscp kekttrkkrn
vnfqkainek lgqyasptak rccqdgvtrl pmmrsceqra
arvqqpdcre pflsccqfae slrkksrdkg qeglgspsar
spa.

SEQ ID NO 6 Protein corresponding to Zn-alpha-2-glycoprotein isolated from Homo sapiens (Accession number in GenBank: GI 38026 CAA42438; 302 amino acids):

mwasmsrmlp vllslllllg pavpqenqdg rysltyiytg
lskhvedvpa fqalgslndl qffrynskdr ksqpmglwrq
vegmedwkqd sqlqkaredi fmetlkdive yyndsngshv
lqgrfgceie nnrssgafwk yyydgkdyie fnkeipawvp
fdpaaqitkq kweaepvyvq rakayleeec patlrkylky
sknildrqdp psvvvtshqa pgekkklkcl aydfypgkid
vhwtragevq epelrgdvlh ngngtyqswv vvavppqdta
pyschvqhss laqplvvpwe as.

### DETAILED DESCRIPTION OF THE INVENTION

The present invention provides an in vitro method for the diagnosis of endometriosis in a subject.

Object of the method described herein is to allow the preferably early diagnosis of endometriosis in female subjects in the absence or in the presence even only of slight symptoms associable to the disease. In particular, it is possible to carry out the diagnosis not only in each one of the conventional four stages of disease, but also before its clinical representation.
In particular, the method is characterized by the determining of the concentration of at least one protein comprising or consisting in an amino acid sequence selected from the group of: SEQ ID NO 1, SEQ ID NO 2, SEQ ID NO 3, SEQ ID NO 4, SEQ ID NO 5 and SEQ ID NO 6 in a biological sample of a subject under examination.
In a preferred embodiment of the present invention, said protein comprises or consists in SEQ ID NO 6. In fact, as mentioned in the foregoing, the Authors of the present invention demonstrated that the variation of the concentration of at least one of the above proteins correlates only with the presence or absence of endometriosis. Specifically, said at least one protein of which the concentration has to be determined is a protein comprising inside its amino acid sequence either SEQ ID NO 1 or SEQ ID NO 2 or SEQ ID NO 3 or SEQ ID NO 4 or SEQ ID NO 5 or SEQ ID NO 6. In other terms, therefore, according to the method described herein, the detectable and/or detected proteins are the proteins (schematically denoted as proteins A-F) below:
   protein A comprising or consisting in SEQ ID NO 1,
   protein B comprising or consisting in SEQ ID NO 2,
   protein C comprising or consisting in SEQ ID NO 3,
   protein D comprising or consisting in SEQ ID NO 4,
   protein E comprising or consisting in SEQ ID NO 5 and/or
   protein F comprising or consisting in SEQ ID NO 6.

In particular, in a preferred embodiment of the invention the detectable and/or detected proteins have an amino acid sequence consisting exclusively in: SEQ ID NO 1 or SEQ ID NO 2 or SEQ ID NO 3 or SEQ ID NO 4 or SEQ ID NO 5 or SEQ ID NO 6.

The detectable and/or detected proteins to the ends of the present invention may be one or two, three, four, five or six and according to any one of the possible combinations. Merely by way of example, when the proteins to be detected (detectable) and/or detected are three, these may be protein A (SEQ ID NO 1), protein D (SEQ ID NO 4) and protein F (SEQ ID NO 5) or protein B (SEQ ID NO 2), protein C (SEQ ID NO 3) and protein F (SEQ ID NO 6), or again protein A (SEQ ID NO 1), protein C (SEQ ID NO 3) and protein F (SEQ ID NO 6). Apparently, any possible combination of the 6 proteins defined above may be utilized to the ends of the present method and, therefore, is as such comprised within the protective scope of the invention described herein.
Moreover, comprised within the protective scope defined herein are also mutated sequences and/or post-translational variants of the sequences defined by SEQ ID NO 1-6.

“Mutated sequences” in the present invention signifies an amino acid sequence X having, with respect to the amino acid sequence indicated in SEQ ID NO 1 or SEQ ID NO 2 or SEQ ID NO 3 or SEQ ID NO 4 or SEQ ID NO 5 or SEQ ID NO 6, at least 90% homology. In other terms, at least the 90, 91, 92, 93, 94, 95, 96, 97, 98, 99% of the amino acid sequence could be identical to each of the above-defined SEQ IDs.

“Post-translational variants” instead signifies amino acid sequences differing from the sequences SEQ ID NO 1-6, or mutants thereof, for the presence of one or more amino acids on which functional groups like, by way of a non-limiting example, glucidic groups, phosphate, acetyl have been added.

Accordingly, in the carrying out of the method described herein, proteins comprising or consisting in mutated sequences and/or post-translational variants of SEQ ID NO 1-6 may also be detected.

The determining of the concentration of the above proteins can be performed according to any one of the methods deemed suitable therefor by the technician in the field. Such methods are widely known in the literature and described in detail in most laboratory manuals, therefore it is not necessary to further delve into them herein.

Merely by way of example, the concentration of the above proteins in a sample, with respect to a control sample, can be determined by quantitative and semiquantitative immunometric methods. In particular, such methods are based on the recognizing by specific antibodies of the sequences represented by SEQ ID NO 1-6.

The development of an antibody capable of selectively recognizing a given amino acid sequence by now falls within conventional laboratory techniques; in fact, said development is not only described in laboratory manuals, but also performed, upon request, as a service by several biotechnology companies. Therefore, today it is possible to develop an antibody, both of polyclonal and monoclonal type, e.g., against SEQ ID NO 1-6, simply by providing to a company, like e.g. Antibody Resource (http://www.antibodyresource.com/customantibody.html) the sequences of interest in order to obtain a specific antibody for each of SEQ ID NO 1-6.

Preferably, the antibodies developed according to the present invention are monoclonal antibodies.

For completeness of information, let us recall that to make the monoclonal-type primary antibody any standard technique for developing monoclonal antibodies will suffice, like e.g. that defined by Koler and Mirstaein in 1967: It should be briefly recalled herein that each specific antibody, recognizing a specific antigenic determinant (epitope), is produced by a specific B lymphocyte. Isolation and in vitro culture of a cell able to produce a single antibody represents a source of monoclonal antibodies (therefore, monospecific antibodies). However, B lymphocytes, when cultivated in vitro, die after a very short time and therefore cannot be a source for long-term production of antibodies.
Monoclonal antibody technology comprises the isolation of these B lymphocytes, and their subsequent fusion with transformed cells (myelomatous cells), useful for their features of greater growth and survival. Many of the resulting hybrid cells (or hybridomas), which are cultivated in vitro, will retain immortality, besides producing large amounts of the monoclonal antibody.
Fusion between B lymphocytes (coming from the spleen and lymph nodes of an immunized animal) and mouse myeloma (the animal most used), is obtained by intervention of a membrane fusion promoter, like polyethylene glycol.
The medium on which hybrids are grown is of selective type, known under the name of HAT, that, just owing to its composition, inhibits the growth both of myelomas and non-fused spleen cells, but not of the hybridoma completing the two parent lines. Hybridomas are subjected to screening for searching specific antibodies of interest, and those selected are forwarded to storage or mass production.

Antibodies, both polyclonal and monoclonal ones, specific for the above-defined amino acid sequences, are therefore (primary) antibodies which specifically recognize each of the above sequences, and which in turn can then be recognized by a suitable secondary antibody, of course specific for the organism in which the primary antibody has been developed. Such secondary antibody could be labeled, for instance, with any fluorochrome commonly used in secondary antibody labeling, like, e.g., fluorescent substances (by way of example: FITC, Cy3, Cy5, Alexa 488, PEe) or enzymes or substances detectable by enzyme cytochemistry (e.g., radish peroxidase) to thereby allow detecting of the primary antibody and, therefore, of the protein(s) of interest according to conventional detection methods.

Preferably, therefore, the determining of the concentration of at least one of the above-indicated proteins can be performed, by way of example, by western-blot, ELISA (enzyme-linked immunosorbent assay), RIA (radioimmunoassay), immunochemistry or protein arrays.

In particular, a protein array consists of a solid support on which various reagents, among which, e.g., antibodies specific for the proteins described herein, are deposited (“spotted”, in technical jargon) in an orderly manner and at a specific and defined density. Each of these antibodies, by binding its own target protein and thereby isolating it from a complex mixture, such as may be, e.g., a cell lysate, allows, on the basis of protein(antigen)-protein (antibody) interactions occurred, to highlight and quantify the specific protein of interest.

Alternatively, the determining of the concentration of at least one of the proteins described herein can be performed by mass spectrometry.
Next, the value related to the concentration of said at least one protein of interest present in a biological sample of the subject under examination is compared with the control value, i.e. the concentration value obtained for the same protein in a sample belonging to a healthy subject. As it will be apparent to a technician in the field, the control value will preferably be the average value of the concentration of said at least one protein calculated with respect to a cohort of healthy subjects.

According to the method for the diagnosis and/or the evaluation of the risk of developing endometriosis described herein, the variation of the concentration of said at least one protein comprising a sequence selected from the group of: SEQ ID NO 1, SEQ ID NO 2, SEQ ID NO 3, SEQ ID NO 4, SEQ ID NO 5 and SEQ ID NO 6 or mutants and/or post-translational variants thereof in the sample of the subject under examination with respect to the control value will provide information about the presence of endometriosis or the risk of developing endometriosis.

In particular, if at least one protein comprising one among SEQ ID NO 1, SEQ ID NO 4, SEQ ID NO 5 proves to have a statistically lower concentration with respect to the same protein analyzed in the control sample, endometriosis or risk of developing endometriosis will be diagnosed to the subject under examination.

Moreover, if at least one protein comprising one among SEQ ID NO 2, SEQ ID NO 3, SEQ ID 6 proves to have a statistically higher concentration with respect to the same protein in the control sample, endometriosis or risk of developing endometriosis will be diagnosed to the subject under examination.

In a preferred embodiment of the present method, said protein comprises or consists in SEQ ID NO 6.

Therefore, in short, according to the method described herein a decrease of the concentration of said at least one protein comprising one among SEQ ID NO 1, SEQ ID NO 4, SEQ ID NO 5 and/or an increase of the concentration of said protein comprising SEQ ID NO 2, SEQ ID NO 3, SEQ ID NO 6 with respect to said control is indicative of endometriosis or of the risk of developing endometriosis. In particular, by way of example, even the mere observation that only one or two, three among SEQ ID NO 2, SEQ ID NO 3, SEQ ID NO 6 are on the increase, and/or only one or two, three, among SEQ ID NO 1, SEQ ID NO 4, SEQ ID NO 5 are on the decrease with respect to the control, is indicative of the presence of endometriosis.

In order to facilitate the determining of the concentration, preferably, the method described herein can also comprise a preliminary step wherein it is obtained a protein extract from the biological sample to be analyzed.

As highlighted above, the further technical problem solved by the present invention is the development of a noninvasive method for the diagnosis of endometriosis. The Authors have observed a modulation of the expression of the above proteins in serum samples of patients with endometriosis. Therefore, in a preferred embodiment of the invention, the biological sample is represented by a sample of blood or serum of the patient under examination.

Moreover, as evident from the type of pathology at issue, i.e. endometriosis, the patient under examination is a female subject, preferably a human subject. However, any animal in which it is possible to observe endometriosis analogously to a human being, like e.g. in horses, may be considered the “subject” according to what described herein.

A further object of the present description is an in vitro method for the monitoring of endometriosis in a subject. The term “monitoring” herein signifies the control of the pattern of the pathological state of a patient in time. Therefore, “monitoring” means serial controls in time of the quantitative variations of the proteins in a subject with respect to the quantitative values of the same protein(s) in the controls. Preferably, such monitoring may be, without being limited thereto, a monitoring of a therapy against endometriosis. Therefore, object of this case is to evaluate before, during and/or after a generic time interval or a therapeutic pathway, a possible improvement or worsening of the pathological state that, in the specific case in which the patient be subjected to therapy, corresponds to the possible advantage or disadvantage of the prescribed therapy to treat and/or slow down and/or prevent endometriosis.

In particular, said monitoring method comprises the key step of determining the concentration of at least one protein comprising a sequence selected from the group of: SEQ ID NO 1, SEQ ID NO 2, SEQ ID NO 3, SEQ ID NO 4, SEQ ID NO 5 and SEQ ID NO 6. In a preferred embodiment of the present invention said protein comprises or consists in SEQ ID NO 6. Analogously to what already mentioned for the above diagnosis method, the determining of the concentration of said at least one protein may be related both to proteins comprising and consisting exclusively in SEQ ID NO 1-6, as well as to mutants and/or post-translational variants thereof. In particular, the technical aspects related to the proteins to be analyzed, as well as the methodologies for determining protein concentration, useful to the ends of the monitoring method, are to be considered analogous to those already described above for the diagnosis method. Therefore, in particular, the method could further comprise a step of obtaining a protein extract from said biological samples which, preferably, are samples of blood or serum.

The determining of the protein concentration to the ends of the monitoring should be performed in a first biological sample and in at least one second biological sample of a subject, obtained respectively at a time t=0 and t>0. The subject under examination may be, in particular, both a subject undergoing therapy against endometriosis and a subject monitored in time without necessarily undergo any type of therapy. In other terms, therefore, the first biological sample obtained at time t=0 may be a sample acquired, in a subject undergoing therapy against endometriosis, e.g., before the start of the therapy itself, and instead in a subject not undergoing therapy, acquired at a generic time t=0a. Otherwise, the second biological sample may be acquired at one or more time intervals from said time t=0, therefore defined as times t>0, e.g., every 15, 20, 30 days. Preferably, the first and second sample are respectively obtained prior to initiation of a therapy against endometriosis and during and/or after said therapy. Then, a comparison between the concentration obtained in said first and said at least second sample for a same protein, among those indicated herein, will provide information about the course of the patient's pathological state.

The variation of the concentration of at least one of the proteins comprising or consisting in SEQ ID NO 1-6 between the two samples analyzed is, therefore, the instrument allowing to the clinician an evaluation of the effectiveness or non-effectiveness of the selected therapeutic strategy. In particular, a decrease of the concentration of said at least one protein comprising or consisting in SEQ ID NO 1, SEQ ID NO 4 or SEQ ID NO 5 and/or an increase of the concentration of said protein comprising or consisting in SEQ ID NO 2, SEQ ID NO 3 or SEQ ID NO 6 in said first sample with respect to said at least second one is indicative of a progression of endometriosis, in other terms therefore of a scarcely effective therapy. Vice versa, an increase of the concentration of said at least one protein comprising or consisting in SEQ ID NO 1, SEQ ID NO 4 or SEQ ID NO 5 and/or a decrease of the concentration of said protein comprising or consisting in SEQ ID NO 2, SEQ ID NO 3 or SEQ ID NO 6 in said first sample with respect to said at least second one is indicative of the effectiveness of the therapy against endometriosis. Alternatively, a non-variation of the concentration of the above proteins in said samples might indicate a slowing down and/or stopping of the progression of endometriosis in the patient.

As already highlighted, the type of therapy to be monitored is a generic therapy against endometriosis. In particular, the therapy may also be a surgical-type treatment.

Object of the present description is also a kit for the diagnosis of endometriosis and/or the monitoring of endometriosis in a subject. Preferably, the kit may be a kit for the monitoring of a therapy against endometriosis. In particular, said kit comprises at least one aliquot of one or more reagents necessary to the determining of the concentration of at least one protein comprising or consisting in a sequence selected from the group of: SEQ ID NO 1, SEQ ID NO 2, SEQ ID NO 3, SEQ ID NO 4, SEQ ID NO 5 and SEQ ID NO 6, or mutants and/or post-translational variants thereof, in a biological sample of said subject. In a preferred embodiment of the present invention, said one or more reagents are necessary to the determining of the concentration of a protein comprising or consisting in SEQ ID NO 6.

Preferably, the kit according to the invention could contain one or more aliquots of at least one specific primary monoclonal or polyclonal antibody against one of the sequences defined in SEQ ID NO. 1, SEQ ID NO. 2, SEQ ID NO. 3, SEQ ID NO. 4, SEQ ID NO. 5 or SEQ ID NO. 6. The kit could also contain one or more aliquots of a labeled or unlabeled secondary antibody, said secondary antibody being of course specific for the immune system from which the primary antibody was made. Therefore, if the primary antibody is made in mouse, the secondary one will be anti-mouse, if made in mouse will be anti-rabbit, etc.

The kit could further contain negative controls and/or positive controls. In particular, the kit can contain as positive control one or more aliquots comprising one or more amino acid sequences selected from the group of: SEQ ID NO 1, SEQ ID NO 2, SEQ ID NO 3, SEQ ID NO 4, SEQ ID NO 5 and SEQ ID NO 6.
Moreover, the kit may comprise suitable reagents and means for the procedure of detecting the above proteins by use of antibodies, such as the PBS buffer or other reagents commonly used for antibody detection.
The following examples and experimental results have the purpose of indicating ways of embodiment of the present description, without however being limitative thereof.

### EXAMPLES

### Example 1

### Biological Material Collection and Storage

Ten plasma samples, respectively from 5 patients with ascertained endometriosis, after serum collection with surgery and histological examination, and from 5 healthy control patients, were collected and stored at the temperature of −80° C. In detail, blood collections performed by peripheral vein pricking were centrifuged at 3.000 rpm for 10 minutes at 4° C., and the plasma obtained aliquoted and stored at −80° C.

### Example 2

### Proteomics Analysis by Two-Dimensional Gel Method

The plasma samples were depleted in advance of the most abundant proteins present in the serum (albumin, IgG, antitrypsin, IgA, transferrin and aptoglobine), so as to be able to highlight with greater clarity proteins differently expressed in the two groups of patients. Then, proteins were extracted from 10 plasma samples and said proteins were separated by electrophoresis on two-dimensional gels (20×30 cm 2DE gel). The proteins were highlighted on the gel by a silver-based stain, suitable to a subsequent Mass Spectrometry application. By use of dedicated software, images of the various gels were compared to single out spots expressed in a constantly and significantly different manner in the two groups of patients. Appropriate statistic tests were used to confirm the statistic validity of these differences. This analysis singled out 5 spots significantly and constantly expressed in a different manner in patients with endometriosis with respect to healthy control patients.
By a mass spectrometry method (LC ESI MS/MS), the proteins corresponding to such differentially expressed spots were singled out. Identification of the proteins corresponding to the spots identified by 2D gel was performed by nanoLC-ESI-MS/MS technology. The MS apparatus was a system of an Agilent 1100 nanoLC system (Agilent, Waldbronn, Germany), a NanoMate 100 (Advion, Ithaca, USA) and a Finnigan LTQ-FT mass spectrophotometer (ThermoFisher, Bremen, Germany). Protein spots were digested directly in-gel with trypsin (Promega, Mannheim, Germany) and applied to the nanoLC-ESI-MS/MS system. Peptides produced by protein spot digestion were trapped on a specific enrichment column (Zorbax SB C18, 0.3×5 mm, Agilent) for five minutes, using 1% acetonitrile0.5% formic acid as eluent; then, the peptides were separated on a Zorbax 300 SB C18 column, 75 μm×150 mm (Agilent) by using a gradient of 0.1% acetonitrile/formic acid from 5% to 40% of acetonitrile for a 40-min period. Mass spectra were automatically recorded by mass spectrometer, following the use conditions indicated by the manufacturer for nanoLC-ESI-MS/MS analyses. The corresponding proteins were then identified by using the MS/MS research system of Mascot search engine (Matrix Science, London, England) and the appropriate protein database (National Center for Biotechnology Information, Bethesda, USA).

The proteins singled out through said method are respectively:

1) Protein corresponding to human apolipoprotein E mutant E3K (SEQ ID NO:1);

2) Protein corresponding to chain A of human antithrombin Iii complex (SEQ ID NO:2);

3) Protein corresponding to chain A of human serum albumin (SEQ ID NO:3);

4) Protein corresponding to precursor C3 of the complement (SEQ ID NO:4);

5) Protein isolated from Homo sapiens (SEQ ID NO:5);

6) Protein corresponding to glycoprotein Zn-alpha 2 (SEQ ID NO:6)

In FIGS. 1A-F are shown the histograms showing the different expression of the above proteins in a serum sample of patients with endometriosis, with respect to serum samples of a control population.

### REFERENCE

Fassbender A, Waelkens E, Verbeeck N, Kyama CM, Bokor A, Vodolazkaia A, Van de Plas R, Meuleman C, Peeraer K, Tomassetti C, Gevaert O, Ojeda F, De Moor B, D'Hooghe T: Proteomics analysis of plasma for early diagnosis of endometriosis. Obstet Gynecol 2012, 119: 276-285.

Giudice L C, and Kao L C: Endometriosis. The Lancet, 364: 1789-1799, 2004.

Hadfield R, Mardon H, Barlow D, Kennedy S. Delay in the diagnosis of endometriosis: a survey of women from the USA and the UK. Hum Reprod 1996, 11: 878-880.

Houston D E: Evidence for the risk of pelvic endometriosis by age, race, and socioeconomic status. Epidemiol Rev, 6: 167-191, 1984.

May K E, Conduit-Hulbert S A, Villar J, Kirtkley S, Kennedy S H, Becker C M: Peripheral biomarkers of endometriosis: a systematic review. Hum Reprod 2010, 16: 651-674.

1. An in vitro method for diagnosis of endometriosis in a subject, comprising the following steps:
a) determining concentration of SEQ ID NO: 6, or mutants and/or post-translational variants thereof, in a biological sample of said subject and in a control sample; and
b) comparing said concentration between said biological sample and said control sample;
wherein an increase of the concentration of said SEQ ID NO: 6 with respect to SEQ ID NO: 6 in said control is indicative of endometriosis.

2. The method according to claim 1, wherein said step a) further comprises determining concentration of at least one protein comprising a sequence selected from the group consisting of: SEQ ID NO: 5, SEQ ID NO: 4, SEQ ID NO: 3, SEQ ID NO: 2, SEQ ID NO:1, mutants, and post-translational variants thereof, in a biological sample of said subject; and
wherein an increase of the concentration of said at least one protein comprising a sequence of SEQ ID NO: 3, or SEQ ID NO: 2 and/or a decrease of the concentration of said at least one protein comprising a sequence of SEQ ID NO: 5, SEQ ID NO: 4, or SEQ ID NO: 1 with respect to the same protein in said control is indicative of endometriosis.

3. The method according to claim 1, further comprising a step of obtaining a protein extract from said biological sample.

4. The method according to claim 1, wherein said biological sample is blood or serum.

5. The method according claim 1, wherein said determining is performed by western-blot, ELISA (enzyme-linked immunosorbent assay), RIA (radioimmunoassay), immunochemistry, protein arrays, and/or mass spectrometry.

6. An in vitro method for monitoring of endometriosis in a subject, comprising the following steps:
a) determining concentration of at least one protein comprising SEQ ID NO: 6or mutants and/or post-translational variants thereof, in a first biological sample and in a second biological sample, obtained respectively at a time t=0 and t>0, of said subject; and
b) comparing said concentration between said first biological sample and said second biological sample.

7. The method according to claim 6, wherein said step a) further comprises determining concentration of at least one protein comprising a sequence selected from the group consisting of: SEQ ID NO: 5, SEQ ID NO: 4, SEQ ID NO: 3, SEQ ID NO: 2, SEQ ID NO: 1, mutants thereof, and post-translational variants thereof, in a biological sample of said subject.

8. The method according to claim 7, wherein an increase of the concentration of said at least one protein comprising a sequence SEQ ID NO: 6, SEQ ID NO: 3, or SEQ ID NO: 2 and/or a decrease of the concentration of said at least one protein comprising a sequence of SEQ ID NO: 5, SEQ ID NO: 4, or SEQ ID NO: 1 in said first sample with respect to said second sample is indicative of progression of endometriosis.

9. The method according to claim 6, wherein said first biological sample and said second biological sample are respectively obtained prior to initiation of a therapy and during and/or after said therapy.

10. The method according to claim 6, further comprising a step of obtaining a protein extract from said biological samples.

11. The method according to claim 6, wherein said biological samples are blood or serum.

12. The method according to claim 9, wherein said therapy is a surgical treatment.

13. A kit for the diagnosis of endometriosis and/or for the monitoring of endometriosis in a subject, comprising:
at least one aliquot of one or more reagents necessary for determining concentration of at least one protein comprising SEQ ID NO: 6or mutants and/or post-translational variants thereof, in a biological sample of said subject.

14. The kit according to claim 13, further comprising at least one aliquot of one or more reagents necessary for determining of concentration of at least one protein comprising a sequence selected from the group consisting of: SEQ ID NO: 5, SEQ ID NO: 4, SEQ ID NO: 3, SEQ ID NO: 2, SEQ ID NO: 1, mutants thereof, and post-translational variants thereof, in a biological sample of said subject.

15. The kit according to claim 13, further comprising at least one aliquot of a positive control comprising one or more amino acid sequences selected from the group consisting of: SEQ ID NO: 6, SEQ ID NO: 5, SEQ ID NO: 4, SEQ ID NO: 3, SEQ ID NO: 2, and SEQ ID NO: 1.
